SEQUENCE LISTING (4 sequences)

SEQ ID NO: 1 - DNA, 2790 bp, Artificial
  Construct comprising autotransporter domain and NADH oxidase
atggttaaattaaaatttggtgttttttttacagttttactatcttcagcatatgcacatggaacacctcaaaatattactgatttgctcgagaaagtcacagttgttggttgtacacatgccggaacctttgcgattaaacaaatcttggccgaacaccctgatgccgaagtgaccgtctacgaacgtaacgatgtcatttcatttctctcttgtggaatcgccctttacctggggggaaaagtcgctgatccgcaaggcctcttttattcaagtcctgaagaactccaaaaattaggcgctaatgtccaaatgaatcacaatgttttagcgatcgatcctgatcaaaagacagtgaccgttgaggacttaaccagtcatgcacaaacgactgagtcatacgataaactagtcatgacgtctggttcttggccaattgtccccaagattccgggcatcgatagcgatcgcgttaagctctgcaaaaactgggcacatgcgcaagctctaatcgaagatgctaaggaagccaagcggattaccgttattggtgccggctatattggtgctgaactagcagaagcctactccactactggtcatgacgtaaccttaattgatgcgatggaccgggttatgcccaagtactttgatgctgattttacggatgtcattgaacaagattatcgggatcacggtgtccaacttgccttaagtgaaacggttgaaagctttactgatagtgcaactgggttgaccattaagactgataagaacagctatgaaacggatctcgctattttatgcattggctttagaccaaataccgacctgctgaaaggcaaagtcgatatggcaccaaacggcgcgattattacggatgactacatgcgttcttctaaccctgatattttcgccgctggtgacagtgctgctgtgcactacaacccaacccatcagaatgcttacattcccttagcaactaacgcggtgcgtcaaggtatcctagtcggtaaaaacctagttaagccgaccgtcaagtatatgggaacacaatcatcttctggtttggcactctatgatcggacgatcgtctcaactggtttaacgctagcagctgcaaaacaacaaggggtgaacgctgaacaagtgattgttgaagataattatcgcccagagtttatgccgtcaactgaacccgttttgatgtcattagtctttgaccccgacacacaccggatcttaggtggtgcgttaatgagtaaatacgatgtttcacaatcggccaacaccctttctgtttgcatccaaaacgaaaatacaattgatgacttagcgatggttgatatgctcttccagcctaactttgatcgaccattcaactacctaaacatcttagcgcaagctgctcaggcgaaagttgcccaatcagttaacgctggtacccttaatcctacaaaagaaagtgcaggtaatactcttaccgtgtcaaattatactgggacaccgggaagtgttatttctcttggtggtgtgcttgaaggagataattcacttacggaccgtctggtggtgaaaggtaatacctctggtcaaagtgacatcgtttatgtcaatgaagatggcagtggtggtcagacgagagatggtattaatattatttctgtagagggaaattctgatgcagaattctctctgaagaaccgcgtagttgccggagcttatgattacacactgcagaaaggaaacgagagtgggacagataataagggatggtatttaaccagtcatcttcccacatctgatacccggcaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttctga

SEQ ID NO: 2 - protein, 929 aa, Artificial
  Construct comprising autotransporter domain and NADH oxidase
MVKLKFGVFFTVLLSSAYAHGTPQNITDLLEKVTVVGCTHAGTFAIKQILAEHPDAEVTVYERNDVISFLSCGIALYLGGKVADPQGLFYSSPEELQKLGANVQMNHNVLAIDPDQKTVTVEDLTSHAQTTESYDKLVMTSGSWPIVPKIPGIDSDRVKLCKNWAHAQALIEDAKEAKRITVIGAGYIGAELAEAYSTTGHDVTLIDAMDRVMPKYFDADFTDVIEQDYRDHGVQLALSETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGVNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNAGTLNPTKESAGNTLTVSNYTGTPGSVISLGGVLEGDNSLTDRLVVKGNTSGQSDIVYVNEDGSGGQTRDGINIISVEGNSDAEFSLKNRVVAGAYDYTLQKGNESGTDNKGWYLTSHLPTSDTRQYRPENGSYATNMALANSLFLMDLNERKQFRAMSDNTQPESASVWMKITGGISSGKLNDGQNKTTTNQFINQLGGDIYKFHAEQLGDFTLGIMGGYANAKGKTINYTSNKAARNTLDGYSVGVYGTWYQNGENATGLFAETWMQYNWFNASVKGDGLEEEKYNLNGLTASAGGGYNLNVHTWTSPEGITGEFWLQPHLQAVWMGVTPDTHQEDNGTVVQGAGKNNIQTKAGIRASWKVKSTLDKDTGRRFRPYIEANWIHNTHEFGVKMSDDSQLLSGSRNQGEIKTGIEGVITQNLSVNGGVAYQAGGHGSNAISGALGIKYSF

SEQ ID NO: 3 - DNA, 28 bp, Artificial
  Forward primer used to clone NADH oxidase
atctcgagaaagtcacagttgttggttg

SEQ ID NO: 4 - DNA, 25 bp, Artificial
  Reverse primer used to clone NADH oxidase
atggtaccagcgttaactgattggg

(19) United States
(12) Patent Application Publication
(10) Pub. No.: US 2013/0203118 A1
(43) Pub. Date: Aug. 8, 2013

(54) Whole-Cell Biocatalyst

(71) Applicants: Joachim Jose, Dusseldorf (DE); Ruth Maas, Dusseldorf (DE); Eva Kranen, Dusseldorf (DE)

(72) Inventors: Joachim Jose, Dusseldorf (DE); Ruth Maas, Dusseldorf (DE); Eva Kranen, Dusseldorf (DE)

(73) Assignee: Zyrus Beteiligungsgesellschaft MBH & Co., Patente I KG, Schonefeld/Waltersdorf (DE)

(21) Appl. No.: 13/818,942

(22) PCT Filed: Aug. 26, 2011
(86) PCT No.: PCT/EP2011/064760

§ 371 Date: Apr. 9, 2013

(30) Foreign Application Priority Data
Aug. 27, 2010 (DE) 10 2010 035 702.2

(51) Int. Cl. C12N 9/00; C12P 19/36; C12P 1/04; C12N 15/70

(52) U.S. Cl. CPC C12N 9/0036; C12N 15/70; C12P 19/36; C12P 1/04
USPC 435/90; 435/320.1; 435/189; 435/190; 435/252.33; 435/471; 435/170

(57) ABSTRACT

The invention relates to a nucleic acid molecule comprising a section that encodes a signal peptide, a section that comprises a heterologous redox factor-regenerating polypeptide, an optional section that encodes a protease detection site, a section that encodes a transmembrane linker, and a section that encodes a transporter domain of an autotransporter or a variant thereof. The nucleic acid molecule enables the expression of redox factor-regenerating enzymes.

The invention relates to a nucleic acid molecule, comprising (1) a segment coding for a signal peptide, (2) a segment comprising a heterologous redox factor-regenerating polypeptide or a variant thereof, (3) optionally a segment coding for a protease recognition site, (4) a segment coding for a transmembrane linker and (5) a segment coding for a transporter domain of an autotransporter or a variant thereof.

About a quarter of all known enzymes are oxidoreductases. A wide range of applications has already been investigated for this group of enzymes, including the synthesis of chiral compounds such as alcohols, aldehydes and amino acids, the production and modification of polymers, the production of biosensors for the most varied applications and the degradation of harmful substances. Oxidoreductases are characterized in that they comprise not only at least one polypeptide chain, but also one or more redox-reactive groups, which can be one or more redox-reactive amino acid side chains or prosthetic groups.

The substrates of the oxidoreductases include metabolites and redox factors. In contrast to prosthetic groups of enzymes, these substrate redox factors do not act as catalysts, but participate in the reaction in stoichiometric amounts and enter into chemical reactions with other substrates. In view of the fact that redox factors are in general very much more expensive than other organic substrates, the feasibility of industrial syntheses using oxidoreductases is limited because of the need to provide suitable reduced or oxidized factors. For solving this problem, scientists have suggested using redox factor-regenerating enzymes. For example, the use of formate dehydroxygenase (FDH) and NADH oxidase or the use of glucose dehydrogenase (GDH) has been proposed for the regeneration of NADH or NADPH. It should be possible to immobilize such enzymes on suitable carriers for several catalysis cycles, to avoid having to repeatedly purify large amounts of the polypeptides.

Unfortunately redox factor-regenerating enzymes are often unstable, especially when they are in the form of immobilized enzymes.

Sanjust et al. (1997) immobilized the NADH oxidase from Thermus aquaticus on various carriers and compared the kinetic properties of soluble and immobilized enzymes. It was found that the immobilized enzyme has lower thermal stability than the soluble enzyme. Hummel and Riebel (2003) describe the use of soluble, purified NADH oxidase from Lactobacillus brevis for regenerating NADH oxidase. They demonstrate that the enzyme has a tendency toward rapid oxidative inactivation, most probably owing to the presence of a cysteine residue that is susceptible to oxidation in the catalytic center of the protein. Therefore the use of a reducing agent (1 mM DTT or mercaptoethanol) is described as being important for maintaining the catalytic capacity of the enzyme. Ahmed and Claiborne (1992) purified the FAD-containing NADH oxidase from Streptococcus faecalis 10C1 and teach that the presence of 2 mM DTT is necessary for stabilizing the holo-oxidase. El-Zahab et al. (2004), verified by Lee and Ping (2007), describe the immobilization of redox factor-regenerating enzymes, for example glucose dehydrogenase (GDH), on a solid support, in the narrower sense on nanoporous silica glass. They do not, however, describe how said enzymes can be stabilized.

Therefore the object of the present invention is to provide an agent with redox factor-regenerating activity, wherein the source of the activity is not only easily accessible for substrate molecules, but can easily be amplified, recycled and regenerated, to make several catalytic steps possible, without the necessity of repeatedly producing fresh agent. Another object of the present invention is to provide an agent that has, in itself or in the presence of potentially inactivating chemicals, such as inhibiting metal ions, or at acidic or basic pH, a redox factor-regenerating activity with better properties than the original form of the corresponding enzyme with respect to kinetics, thermal stability, storage capability and stability. A particular object of the present invention is to provide an agent that displays stable redox factor-regenerating activity in the presence of oxygen or of oxygen-releasing agents or in oxidizing conditions, whose stability is better than that of the original form of the corresponding enzyme.

The present inventors found, surprisingly, that using the autotransporter system, redox factor-regenerating enzymes can be expressed on the surface of a whole cell and in themselves are surprisingly stable with respect to the presence of oxygen and different temperatures and conditions. Moreover, the present inventors found, surprisingly, that redox factor-regenerating enzymes can be incubated and remain stable for quite a long time in various solutions and buffers in various conditions.

Autodisplay is an elegant tool for presenting recombinant proteins on the surface of bacteria. This expression system is based on the secretion mechanism of the protein family of autotransporters belonging to the type V secretion system.

In Gram-negative bacteria, the autotransporter pathway formed both for the transport of proteins to the cell surface and for the secretion of proteins into the extracellular space (Jose and Meyer, 2007). The autotransporter proteins are synthesized as precursor proteins, which fulfill all structural requirements for transport to the cell surface (Jose, 2006). They are synthesized with an N-terminal signal peptide, which is typical of the Sec pathway, which makes crossing the inner membrane possible. Once inside the periplasm, after truncation of the signal peptide the C-terminal moiety of the precursor folds into the outer membrane as a porin-like structure, a so-called β-barrel. Through this pore, the N-terminal-bound passenger domain is translocated to the surface (Jose et al., 2002). There it can be cleaved off—either autoproteolytically or by an additional protease—or can remain anchored via the transporter domain on the cell envelope. Replacing the natural passenger with a recombinant protein leads to its surface translocation. For this, using genetic engineering techniques it is necessary to construct a nonnatural precursor, consisting of a signal peptide, the recombinant passenger, the β-barrel and a linker region in between, which is required for unrestricted access to the surface. In this way, the AIDA-I autotransporter has already been used successfully for efficient surface display of various passenger domains (Henderson et al., 2004). With the autodisplay system, self-association of subunits on an active enzyme has been observed, for example with the dimeric enzyme sorbitol dehydrogenase (Jose, 2002; Jose and von Schwichow, 2004).

In particular, autodisplay technology is a method of expression for certain proteins on the surface of the outer membrane of E. coli and other Gram-negative bacteria, wherein the autodisplay system is based on the natural secretion mechanism of the autotransporter proteins (A. Banerjee et al. (2002)). The transport of the recombinant passenger protein can take place simply by insertion, in the reading frame, of its coding sequence between the signal peptide and the translocating domain of the autodisplay vector using usual genetic engineering techniques. The signal peptide can be obtained from a subunit of the cholera toxin, and it can be combined with a nonnatural promoter. Consequently, the passenger protein that is to undergo translocation through the outer membrane is expressed as recombinant fusion protein with another protein, called autotransporter, on the outer membrane of E. coli (AIDA-I) (Jose, 2006). The C-terminal moiety of the autotransporter protein forms a porin-like structure (β-barrel) within the outer membrane of E. coli. This porin-like structure enables the recombinant passenger protein to be translocated to the surface of the outer membrane of E. coli (Jose, 1995, 2006, 2007).

The concept of immobilization of redox cofactor-regenerating polypeptides dates back to the year 1974, when Sarborsky and Ogletree described the immobilization of glucose oxidase via activation of its carbohydrate residues. The concept of the autodisplay system, which was first described by Jose et al. (1995), has also been known for more than 15 years. Nevertheless, the use of the autotransporter system for immobilizing redox factor-regenerating enzymes is neither taught, nor proposed by the prior art, to the best of our knowledge.

The object of the present invention is solved by the objects of the independent claims. Preferred embodiments can be found in the dependent claims.

The object of the present invention is, according to a first aspect, which is in addition the first embodiment of the first aspect, solved with a nucleic acid molecule, comprising (1) a segment coding for a signal peptide, (2) a segment comprising a heterologous redox factor-regenerating polypeptide or a variant thereof, (3) optionally a segment coding for a protease recognition site, (4) a segment coding for a transmembrane linker and (5) a segment coding for a transporter domain of an autotransporter or a variant thereof.

In a preferred embodiment of the present invention, the nucleic acid molecule is linked functionally to an expression regulating sequence. In a preferred embodiment, the term “expression regulating sequence”, as used here, refers to a nucleic acid sequence that can regulate the expression level of a nucleic acid molecule, preferably downstream of the expression regulating sequence. The expression regulating sequence can for example be a promoter. A person skilled in the art is familiar with sequences suitable for regulating expression and methods for functionally linking these sequences to a nucleic acid molecule.

In a preferred embodiment of the present invention, the nucleic acid molecule is part of a recombinant plasmid. In a preferred embodiment the nucleic acid molecule comprises SEQ ID NO: 1 or variants thereof.

In a preferred embodiment of the present invention the term “heterologous”, as used here, refers to a nucleic acid that was constructed using genetic engineering techniques, for example by joining together an expression regulating sequence and a sequence to be expressed, which is not normally under the control of this expression regulating sequence, or by using a sequence that has a point mutation relative to the original sequence. Even if only one segment of a construct is designated as heterologous, this consequently implies that the whole construct is heterologous. A person skilled in the art is familiar with genetic engineering techniques. In another preferred embodiment the term “heterologous”, as used here, refers to a nucleic acid coding for a polypeptide, which is joined to an expression regulating sequence, and/or a fused sequence, which is derived from a different organism from the expression regulating sequence and is to be expressed. In another preferred embodiment the term “heterologous”, as used here in connection with a redox factor-regenerating polypeptide, means that the nucleic acid segment that codes for the redox factor-regenerating polypeptide was obtained or taken from another organism as at least one further segment, such as the transporter domain or the expression regulating sequence or the transmembrane linker. For example, the redox factor-regenerating polypeptide is heterologous if it was obtained from Lactobacillus brevis, but all other sequences were obtained from E. coli. In another preferred embodiment the term “heterologous”, as used here, means that the nucleic acid sequence designated as heterologous originates from an organism that is different from the host or intended host that is used for expression or multiplication of this nucleic acid sequence.

In a preferred embodiment of the present invention the term “signal peptide”, as used here, refers to an amino acid sequence, preferably at the N-terminus of a polypeptide, which has the effect that the polypeptide, if it is expressed in the cytosol of a host cell, is translocated to a particular compartment of the cell, preferably a compartment different from the cytosol. In an especially preferred embodiment the host cell is a Gram-negative bacterial cell and the signal peptide has the effect that the forming or finished polypeptide is translocated into the periplasm or the outer membrane of the Gram-negative bacterial cell.

In a preferred embodiment of the present invention the term “protease recognition site”, as used here, refers to a particular amino acid sequence motif in a polypeptide, wherein this sequence motif of said protease is recognized specifically, in such a way that it binds to and cleaves the polypeptide.

In a preferred embodiment of the present invention the term “transmembrane linker”, as used here, refers to a flexible polypeptide segment, which serves for joining the autotransporter domain to the redox factor-regenerating polypeptide, but is sufficiently flexible to allow independent folding and/or transporting of the redox factor-regenerating polypeptide.

In a preferred embodiment of the present invention the term “transporter domain of an autotransporter” refers to a domain that can be used for obtaining the expression product of the nucleic acid molecule, if it is synthesized ribosomally inside the cell, preferably in the bacterial cytoplasm, and is translocated to the outer membrane of the cell, preferably to the side of the outer membrane that is exposed to the surroundings of the cell. In an especially preferred embodiment the transporter domain has the effect that said expression product is located on the external surface of the outer membrane. In a preferred embodiment of the present invention the transporter domain of an autotransporter is a protein that is located on the outer membrane of the cell, and the segment coding for an NADH oxidase is part of a domain, loop or some other part of the transporter domain or is fused thereto, so that the NADH oxidase is presented on the surface of the cell. In another preferred embodiment of the present invention, the transporter domain of an autotransporter is a protein of a system that can be used for displaying polypeptides on the surface of a cell. In an especially preferred embodiment, the transporter domain is a transporter domain of the autodisplay system, also designated as autotransporter pathway, of the AIDA-I type of Gram-negative bacterial cells.

In a preferred embodiment, variants of amino acids or nucleic acid sequences are referred to in the present application explicitly, for example by the name or the deposition number or even by the term “variant of”, or implicitly, for example by a description of the function, in the context of the present invention. In a preferred embodiment the term “variant”, as used here, comprises amino acid or nucleic acid sequences that are 60, 70, 75, 80, 85, 90, 92, 94, 95, 96, 97, 98 or 99% identical to the amino acid serving as reference. In a preferred embodiment the term “variant” comprises, with respect to an amino acid sequence, those amino acid sequences that have a conservative amino acid exchange or several conservative amino acid exchanges relative to the reference sequence. In a preferred embodiment the terms “variants” of an amino acid sequence or nucleic acid sequence comprise active segments and/or fragments of the amino acid sequence or nucleic acid sequence. In a preferred embodiment, the term “active segment”, as used here, refers to an amino acid sequence or a nucleic acid sequence that is shorter than the amino acid or nucleic acid sequence of full length, but still has at least a part of its essential biological activity, e.g. as an NADH oxidase. In a preferred embodiment the term “variant” of a nucleic acid comprises the nucleic acids of the complementary strand, which—preferably under stringent conditions—hybridize to the reference nucleic acid. The stringency of hybridization reactions can readily be determined by a person of average skill in the art and is usually an empirical calculation as a function of the probe length, washing temperature and the salt concentration. In general, longer probes require higher temperatures for perfect annealing, whereas shorter probes require lower temperatures. Hybridization generally depends on the capacity of the denatured DNA for renaturation, when complementary strands are in an environment that has a temperature below the melting point of the strands. The higher the degree of desired homology between the probe and a hybridizable sequence, the higher is the relative temperature that can be applied. It therefore follows that higher relative temperatures tend to make the reaction conditions more stringent, whereas lower temperatures reduce this stringency.

Further details and an explanation of the stringency of hybridization reactions can be found in Ausubel et al. (1995). In another preferred embodiment the term “variant” of a nucleic acid or amino acid refers to a nucleic acid or amino acid that has, at least up to a certain degree, the same biological activity and/or function as the reference nucleic acid or amino acid. In a preferred embodiment, the term “variant” of a nucleic acid sequence, as used here, refers to another nucleic acid sequence, which codes for an amino acid sequence that is similar to the reference amino acid sequence.

In a preferred embodiment of the present invention the term “redox factor”, as used here, refers to an organic compound that is redox-reactive, i.e. that can be oxidized or reduced under physiological conditions. In a preferred embodiment the redox factor does not comprise any redox-reactive amino acid side chains. In a preferred embodiment the redox factor is a free redox factor, i.e. it is not bound covalently to a peptide frame or peptide.

In a preferred embodiment the redox factor has a standard reduction potential of not more than 0.85 (i.e. for high 0.6 V), 0.5, 0.4, 0.2, 0, −0.1, −0.15, −0.2, −0.25, −0.3, or −0.4 V. In an especially preferred embodiment the redox factor has a standard reduction potential from −0.325 to −0.2 V. In a quite especially preferred embodiment the redox factor has a standard reduction potential from −0.35 to −0.3.

In a preferred embodiment, the term “redox factor-regenerating polypeptide”, as used here, refers to a polypeptide that catalyzes the regeneration of a redox factor. In a preferred embodiment, the term “regenerating a redox factor”, as used here, refers to the capacity for reducing an oxidized redox factor, which in its reduced form acts as a reducing agent in a chemical synthesis, or to the capacity for oxidizing a reduced redox factor, which in its oxidized form acts as an oxidizing agent in a chemical synthesis. In another preferred embodiment the term “regenerating a redox factor”, as used here, refers to the restoration of the previous redox state of a redox factor, preferably of the state in which it could be used for a synthesis of interest, especially preferably a synthesis that is catalyzed by an oxidoreductase, wherein the redox factor is used as substrate. For example, NAD⁻ that is consumed in a reaction can be regenerated by oxidizing NADH to NAD⁺.

In a second embodiment of the first aspect of the present invention, which also represents an embodiment of the first embodiment, the redox factor that is regenerated by the redox factor-regenerating polypeptide is selected from the group comprising NADH, NADPH, FADH₂, heme, metal ions, glutathione, pyrroloquinoline-quinone (PQQ), pyridoxal phosphate, thiamine pyrophosphate and ascorbate. Metal ions include, but are not limited to, Fe, Cu, Zn, Ni, Co, Mn, Cr and Mg ions.

In a preferred embodiment each of the aforementioned redox factors has both the oxidized and the reduced form or forms of the corresponding conjugated redox pair, for example both NADH and NAD⁺ in the case of the redox factor NADH, even if only one form is mentioned explicitly.

In a third embodiment of the first aspect of the present invention, which also represents an embodiment of the first and second embodiment of the present invention, the redox factor-regenerating polypeptide comprises one or more flavin cofactors.

In a preferred embodiment, the term “flavin cofactor”, as used here, refers to a redox-reactive cofactor that is based on the isoalloxazine ring system. In a preferred embodiment the flavin cofactor is selected from the group comprising riboflavin, flavin mononucleotide (FMN) and flavin-adenine dinucleotide (FAD), both in their oxidized and reduced forms.

In a fourth embodiment of the first aspect of the present invention, which also represents an embodiment of the first to third embodiment, the redox factor-regenerating polypeptide is selected from the group comprising NADH oxidase, formate dehydrogenase and glucose dehydrogenase.

In a fifth embodiment of the first aspect of the present invention, which also represents an embodiment of the first to fourth embodiment, the redox factor-regenerating polypeptide is selected from the group comprising the NADH oxidases from the genera Lactobacillus, Thermus, Brevibacterium and Streptococcus, preferably the NADH oxidase of Lactobacillus brevis, and variants thereof.

In a sixth embodiment of the first aspect of the present invention, which also represents an embodiment of the first to fifth embodiment, the transporter domain of an autotransporter is selected from the group comprising Ssp, Ssp-h1, Ssp-h2, PspA, PspB, Ssa1, SphB1, AspA/NalP, VacA, AIDA-I, IcsA, MisL, TibA, Ag43, ShdA, AutA, Tsh, SepA, EspC, EspP, Pet, Pic, SigA, Sat, Vat, EpeA, EatA, EspI, EaaA, EaaC, pertactin, BrkA, Tef, Vag8, PmpD, Pmp20, Pmp21, AgA1 protease, App, Hap, rOmpA, rOmpB, ApeE, EstA, Lip-1, McaP, BabA, SabA, AlpA, Aae, NanB and variants thereof.

In another preferred embodiment of the present invention, the term “transporter domain of an autotransporter”, as used here, comprises a domain that displays a polypeptide different from the transporter domain, especially a redox factor-regenerating polypeptide, on the surface of a cell, when said protein has been inserted in or fused with the amino acid sequence of the transporter domain.

Preferably the redox factor-regenerating polypeptide is fused with a transporter domain of an autotransporter. The transporter domain of the autotransporter according to the invention can be any transporter domain of an autotransporter and is preferably capable of forming a β-barrel structure. A detailed description of the β-barrel structure and preferred examples of β-barrel autotransporters are disclosed in WO 97/35 022 and form part of the present description by reference. Henderson et al. (2004) describe autotransporter proteins with suitable autotransporter domains (a summary is given in Table 1 of Henderson et al., 2004). The disclosure of Henderson et al. (2004) forms part of the present description by reference. For example, the transporter domain of the autotransporter can be selected from: Ssp (P09489, S. marcescens), Ssp-h1 (BAA33455, S. marcescens), Ssp-h2 (BAA11383, S. marcescens), PspA (BAA36466, P. fluorescens), PspB (BAA36467, P. fluorescens), Ssa1 (AAA80490, P. haemolytica), SphB1 (CAC44081, B. pertussis), AspA/NalP (AAN71715, N. meningitidis), VacA (Q48247, H. pylori), AIDA-I (Q03155, E. coli), IcsA (AAA26547, S. flexneri), MisL (AAD16954, S. enterica), TibA (AAD41751, E. coli), Ag43 (P39180, E. coli), ShdA (AAD25110, S. enterica), AutA (CAB89117, N. meningitidis), Tsh (154632, E. coli), SepA (CACO5786, S. flexneri), EspC (AAC44731, E. coli), EspP (CAA66144, E. coli), Pet (AAC26634, E. coli), Pic (AAD23953, E. coli), SigA (AAF67320, S. flexneri), Sat (AAG30168, E. coli), Vat (AA021903, E. coli), EpeA (AAL18821, E. coli), EatA (AA017297, E. coli), EspI (CAC39286, E. coli), EaaA (AAF63237, E. coli), EaaC (AAF63038, E. coli), pertactin (P14283, B. pertussis), BrkA (AAA51646, B. pertussis), Tef (AAQ82668, B. pertussis), Vag8 (AAC31247, B. pertussis), PmpD (084818, C. trachomatis), Pmp20 (Q9Z812, C. pneumoniae), Pmp21 (Q9Z6U5, C. pneumoniae), IgAl protease (NP_{—}283693, N. meningitidis), App (CAC14670, N. meningitidis), IgAl1 protease (P45386, H. influenzae), Hap (P45387, H. influenzae), rOmpA (P15921, R. rickettsii), rOmpB (Q53047, R. rickettsii), ApeE (AAC38796, S. enterica), EstA (AAB61674, P. aeruginosa), Lip-1 (P40601, X. luminescens), McaP (AAP97134, M. catarrhalis), BabA (AAC38081, H. pylori), SabA (AAD06240, H. pylori), AlpA (CAB05386, H. pylori), Aae (AAP21063, A. actinomycetemcomitans), NanB (AAG35309, P. haemolytica) and variants of these autotransporters. For each of the examples of autotransporter proteins, examples of suitable GenBank deposition numbers and species, from which the autotransporters can be obtained, are given in parentheses. The transporter domain of the autotransporter is preferably the AIDA-I protein of E. coli or a variant thereof, for example those that were described by Niewert et al. (2001). The AIDA autotransporter system is linked to F18 and Stx2e in E. coli isolates from pigs for which an edema disease and so-called postweaning has been diagnosed.

Variants of the autotransporter sequences given above can be obtained for example by altering the amino acid sequence in the loop structures of the β-barrel that do not belong to the transmembrane segments. Optionally the nucleic acids coding for the surface loops can be deleted entirely. In addition, conservative amino acid exchanges can be made within the amphipathic β-pleated sheet structures, i.e. exchange of a hydrophilic amino acid for another hydrophilic amino acid and/or exchange of a hydrophobic amino acid for another hydrophobic amino acid. Preferably a variant has, at the amino acid level, a sequence identity of at least 70%, at least 80%, at least 90%, at least 95% or at least 98% with the corresponding naturally occurring sequence of the autotransporter domain, especially in the region of the β-pleated sheet structures.

In a seventh embodiment of the first aspect of the present invention, which also represents an embodiment of the first to sixth embodiment, the redox factor-regenerating polypeptide is oxygen-sensitive. In a preferred embodiment the term “oxygen-sensitive”, as used here, means that the corresponding polypeptide undergoes rapid deactivation in the presence of oxygen, preferably at normal concentrations, i.e. occurring in the atmosphere. In a further preferred embodiment the term “oxygen-sensitive”, as used here, means that the corresponding polypeptide loses at least 10, 20, 30, 40, preferably 50, quite especially preferably 80% of its activity, preferably with respect to its k_{cat}, when it is exposed for at least 30 minutes to oxygen at concentrations or under conditions as in the atmosphere, both in solutions and in other phases.

In another embodiment the redox factor-regenerating polypeptide is a soluble polypeptide. In a preferred embodiment, the term “soluble polypeptide “, as used here, refers to a polypeptide that is not bound to a membrane in its natural environment. For example, the NADH oxidase of Lactobacillus brevis, a cytosolic protein, is a soluble polypeptide. In another preferred embodiment the redox factor-regenerating polypeptide is a membrane-bound polypeptide, i.e. a polypeptide that is bound or attached covalently or noncovalently to a cell membrane or an integral membrane protein. For example, complex IV of the respiratory chain is a membrane-bound polypeptide.

According to a second aspect, the object of the present invention is solved with a polypeptide that is encoded by a nucleic acid molecule according to an embodiment of the first aspect of the present invention. In a preferred embodiment the polypeptide comprises SEQ ID NO: 2 or variants thereof.

According to a third aspect, the object of the present invention is solved with a cell that expresses a polypeptide according to the second aspect on its surface or has been transformed using a nucleic acid molecule according to an embodiment of the first aspect of the present invention.

In a preferred embodiment, the term “cell”, which is used interchangeably with the term “host cell”, as used here, refers to a cell that is capable of expressing a polypeptide. Such a cell can also be designated as “whole-cell catalyst or biocatalyst”. In another preferred embodiment the cell or host cell is a prokaryotic cell, preferably a Gram-negative bacterial cell, quite especially preferably an E. coli cell. In another exemplary embodiment the cell is a eukaryotic cell. In another preferred embodiment the cell or host cell is a spore of a prokaryote.

According to a fourth aspect, the object of the present invention is solved with a membrane fraction, which can be obtained from the cell according to the third aspect of the present invention.

A bacterial cell comprises a number of compartments, which are separated from one another by hydrophobic membranes. A Gram-positive bacterial cell has a plasma membrane, which delimits the cytosol, the interior of the cell. The plasma membrane is surrounded by a peptidoglycan layer. In contrast, Gram-negative bacteria possess, in addition to the plasma membrane, another membrane that is called the outer membrane. The term “surface”, as used here, preferably refers to a layer of the microorganism that is exposed to the surroundings, wherein the surroundings are for example the liquid culture medium that is used for culture of the cell of interest. In a quite especially preferred embodiment, a polypeptide according to the present invention is expressed on the outside of the outer membrane of a Gram-negative bacterial cell. In a preferred embodiment a polypeptide according to the present invention is expressed on the inside of the outer membrane of a Gram-negative bacterial cell. In another preferred embodiment the polypeptide according to the invention is expressed on the outside of a spheroplast, wherein it is a Gram-negative bacterial cell from which the outer membrane has been removed. A person skilled in the art is familiar with methods that can be used for producing spheroplasts. In another preferred embodiment the polypeptide according to the invention is expressed on the outside of a Gram-positive bacterial cell. Thus, as the terms “displayed on the surface” and “expressed on the surface” are used here, they are synonymous.

In a preferred embodiment of the present invention, the membrane fraction or the membrane preparation, the two terms being used interchangeably, comprises a redox factor-regenerating polypeptide according to the first aspect of the invention, preferably in a catalytically active state. The terms “membrane fraction” and “membrane preparation”, as used here, preferably refer to a product that is enriched in membrane constituents, preferably constituents of the outer membrane of a Gram-negative bacterium. A person skilled in the art is familiar with protocols and methods that can be used for producing membrane preparations. For example, bacterial cells can be harvested from a culture and submitted to lysis, for example by cycles of freezing and thawing, sonication, resuspension in lysis buffer or the like, followed by differential centrifugation, in order to isolate membrane fractions of the cells. In a preferred embodiment of the present invention, the membrane preparation is a preparation of the outer membrane, i.e. a preparation in which there is enrichment of the constituents of the outer membrane, relative to the constituents of other membranes and compartments, such as the cytosol, the inner membrane and the periplasm. A person skilled in the art is familiar with protocols and methods that can be used for isolating or enriching the outer membrane or constituents thereof, for example a lysozyme treatment of bacterial cells, followed by centrifugation steps. In a preferred embodiment the membrane fraction can be a treated membrane fraction, i.e. the content or the properties of the membrane fraction have been altered, for example by purifying a protein constituent of the membrane fraction or by solubilizing the membrane fractions and/or taking up constituents of the membrane fraction in vesicles. In a preferred embodiment of the present invention, the membrane fraction can be immobilized—for example on the surface of a vessel or a column.

According to a fifth aspect, the object of the present invention is solved with a method of regenerating a redox factor, comprising the following steps: a) providing the polypeptide according to the second aspect, the membrane preparation according to the fourth aspect or the cell according to the third aspect of the present invention and b) contacting the polypeptide according to the second aspect, the membrane preparation according to the fourth aspect or the cell according to the third aspect with at least one substrate of the redox factor-regenerating polypeptide. In a preferred embodiment step a) and/or step b) take place in the absence of a reducing agent and/or in an oxidative environment.

According to a sixth aspect the object of the present invention is solved using the cell according to the third aspect or the membrane fraction according to the fourth aspect or the polypeptide according to the second aspect for regenerating a redox factor.

According to a seventh aspect the object of the present invention is solved by the use of the cell according to the third aspect, the membrane preparation according to the fourth aspect and the polypeptide according to the second aspect of the present invention for adjusting the redox environment of an aqueous solution, preferably by deoxidizing the aqueous solution.

In a preferred embodiment of the present invention the term “adjusting the redox environment of an aqueous solution”, as used here, refers to any measure that acts directly on the redox conditions in an aqueous solution, i.e. the capacity of the solution to oxidize or to reduce a compound. In a preferred embodiment of the present invention the term “deoxidizing an aqueous solution”, as used here, refers to any measure that can be employed for lowering the concentration of oxygen in said solution. A person skilled in the art is familiar with methods that can be used for measuring the oxygen concentration of an aqueous solution, such as, for example, the use of oxygen electrodes.

According to an eighth aspect, the object of the present invention is solved with a method of producing a cell that displays a redox factor-regenerating polypeptide on its surface, comprising: (a) introducing the nucleic acid according to an embodiment of the first aspect of the present invention into a cell and b) optionally contacting the cell with one or more redox-reactive prosthetic groups.

According to a ninth aspect, the object of the present invention is solved with a method of producing the product of a redox factor-dependent polypeptide, comprising the following steps: a) providing a redox factor-dependent enzyme and (b) contacting the redox factor-dependent polypeptide with one or more of its substrates in the presence of the cell according to the third aspect, the membrane preparation according to the fourth aspect and the polypeptide according to the second aspect of the present invention, so that the polypeptide according to the second aspect regenerates the redox factor on which the redox factor-dependent polypeptide depends. In a preferred embodiment, the term “redox factor-dependent polypeptide”, as used here, refers to a polypeptide that has a biological activity, preferably an enzyme activity, which requires a redox factor as cofactor, prosthetic group or, preferably, substrate.

In another preferred embodiment, the object of the present invention is solved with a composition comprising the cell according to the third aspect, the membrane preparation according to the fourth aspect and the polypeptide according to the second aspect of the present invention and a redox factor-dependent polypeptide and the redox factor. In one embodiment the redox factor-dependent polypeptide is displayed using the autotransporter system, i.e. its expression is brought about by a nucleic acid that comprises the following: (1) a segment coding for a signal peptide, (2) a segment comprising a preferably heterologous redox factor-regenerating polypeptide or a variant thereof, (3) optionally a segment coding for a protease recognition site, (4) a segment coding for a transmembrane linker and (5) a segment coding for a transporter domain of an autotransporter or a variant thereof.

The present invention is further illustrated by the following figures and examples that are not to be understood as limiting, from which further features, embodiments, aspects and advantages of the invention can be seen.

FIG. 1 shows the restriction map of the plasmid pAT-NOx.

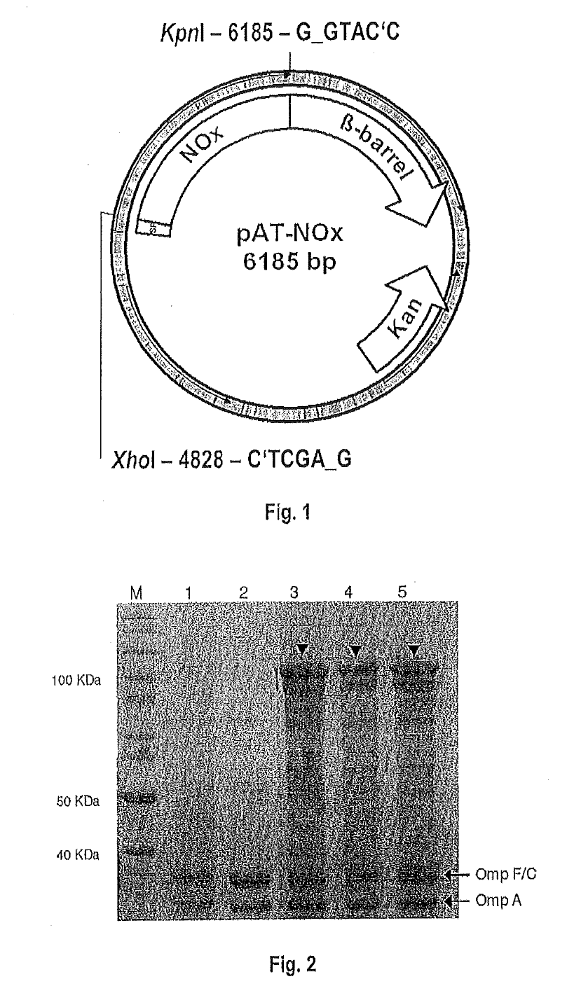

FIG. 2 shows an SDS-gel, which shows NADH oxidase in the outer membrane of E. coli and the orientation through whole-cell digestion. M) molecular weight standard; 1) E. coli UT5600(DE3); 2) E. coli UT5600(DE3) pAT-NOx, not induced; 3) E. coli UT5600(DE3) pAT-NOx, induced; 4) E. coli UT5600(DE3) pAT-NOx, induced, digested with proteinase K; 5) E. coil UT5600(DE3) pAT-NOx, induced, digested with trypsin.

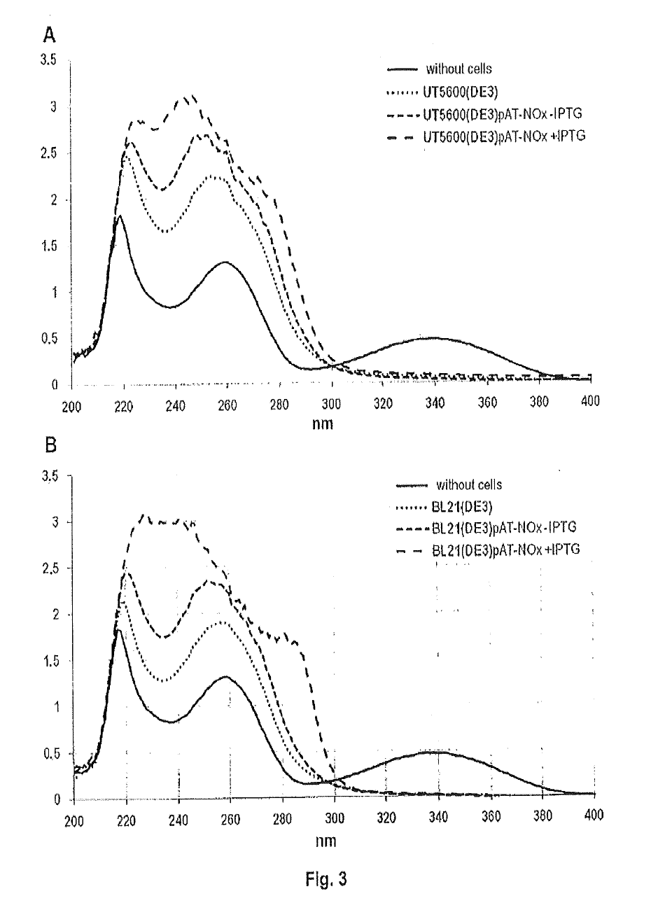

FIG. 3 shows absorption spectra of the supernatant following an NADH oxidase assay using A) E. coli UT5600(DE3) pAT-NOx and B) E. coli BL21(DE3) pAT-NOx, in each case after 1 h induction.

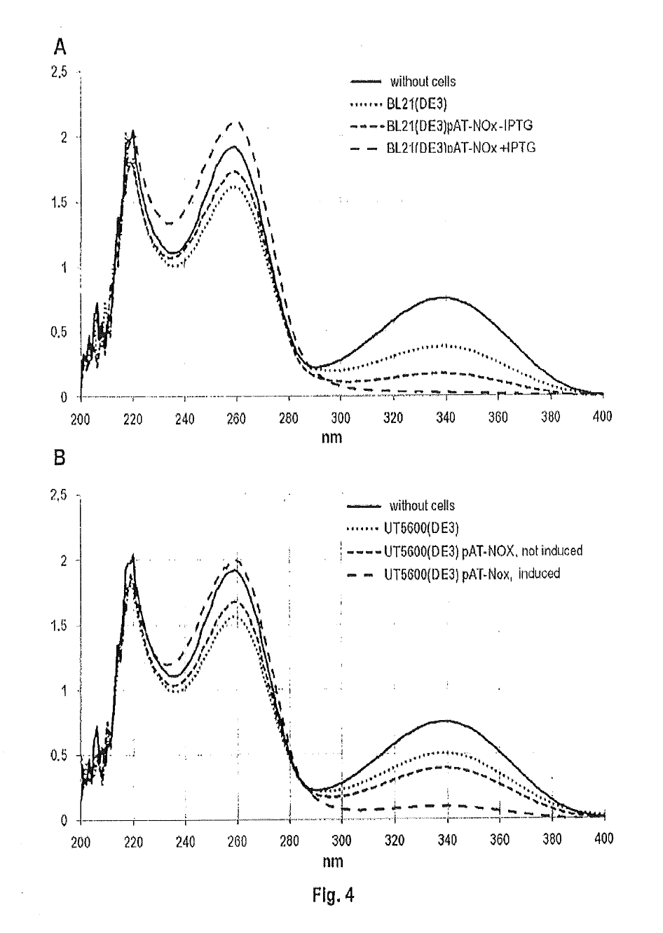

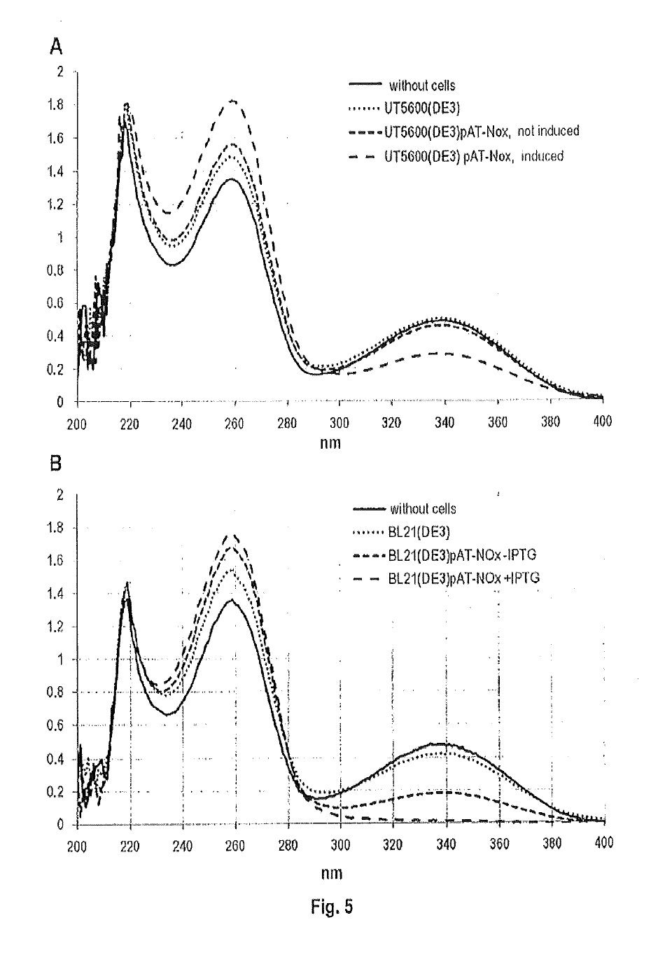

FIG. 4 shows the absorption spectra of the supernatant following an NADH oxidase assay using A) E. coli UT5600(DE3) pAT-NOx and B) E. coli BL21(DE3) pAT-NOx, in each case after 4 h induction.

FIG. 5 shows the absorption spectra of the supernatant following an NADH oxidase assay using A) E. coli UT5600(DE3) pAT-NOx and B) E. coli BL21(DE3) pAT-NOx, in each case after 20 h induction.

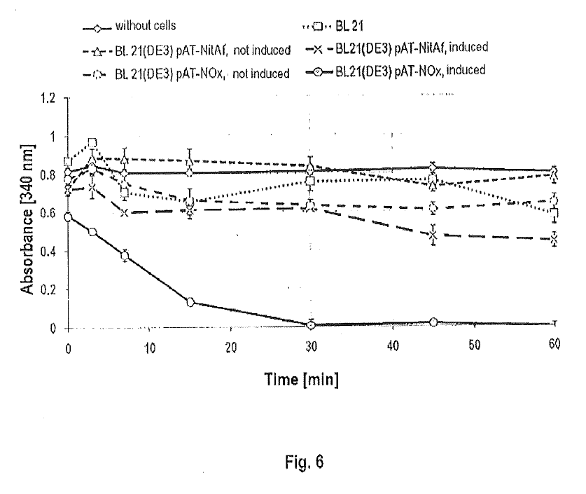

FIG. 6 shows the results of the NADH oxidase assay conducted on microtiter plates using E. coli BL21(DE3) pAT-NOx after growth in an M9 medium. The figure shows the decrease in extinction at 340 nm over time.

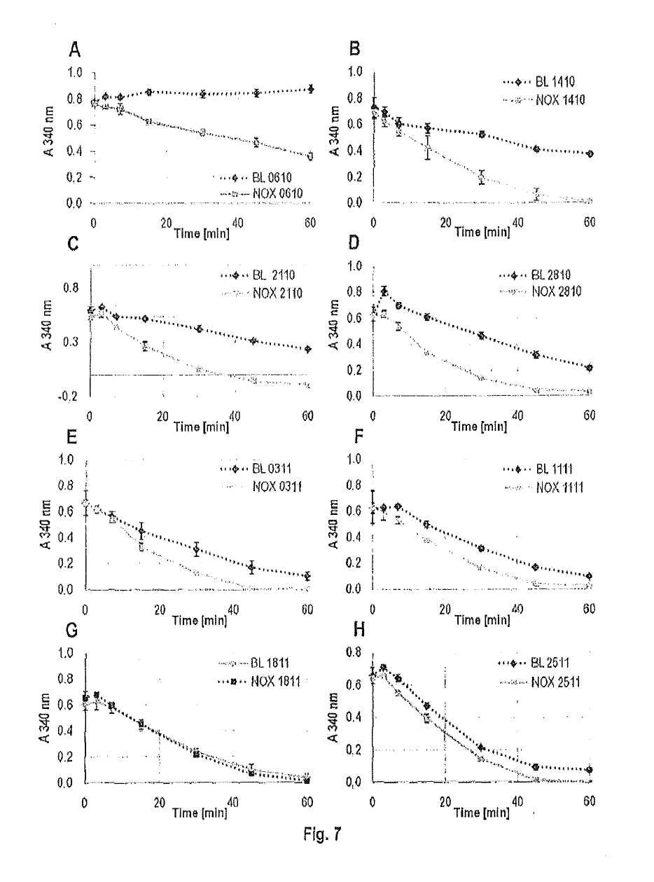

FIG. 7 shows the results for the NOX whole-cell biocatalyst, which had been stored for more than 7 weeks at +8° C. The activity assay was carried out with cells before storage [A] and cells that had been stored for one week [B], two weeks [C], three weeks [D], four weeks [E], five weeks [F], six weeks [G] and seven weeks [H].

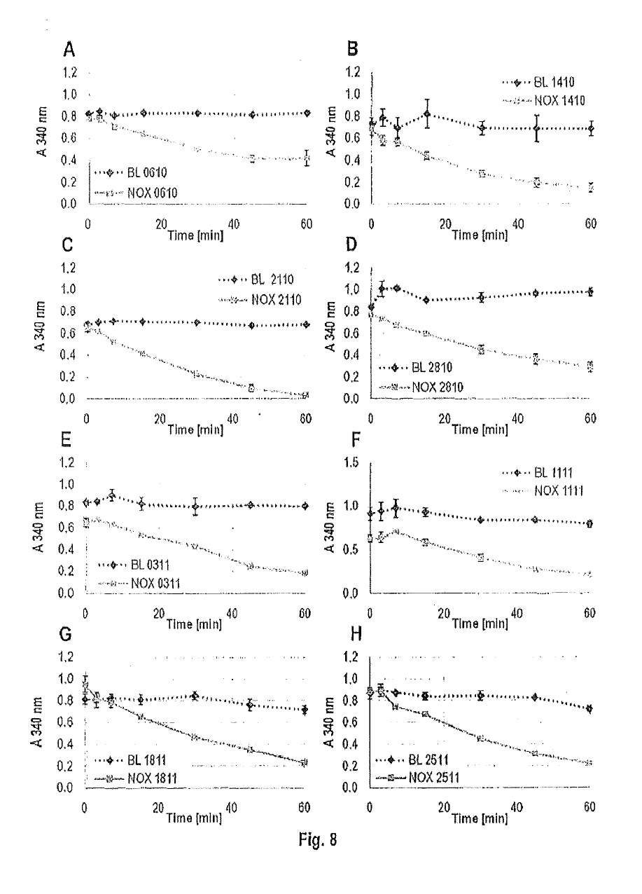

FIG. 8 shows the results for the. NOX whole-cell biocatalyst, which had been stored for more than 7 weeks at −18° C. The activity assay was carried out with cells before storage [A] and cells that had been stored for one week [B], two weeks [C], three weeks [D], four weeks [E], five weeks [F], six weeks [G] and seven weeks [H].

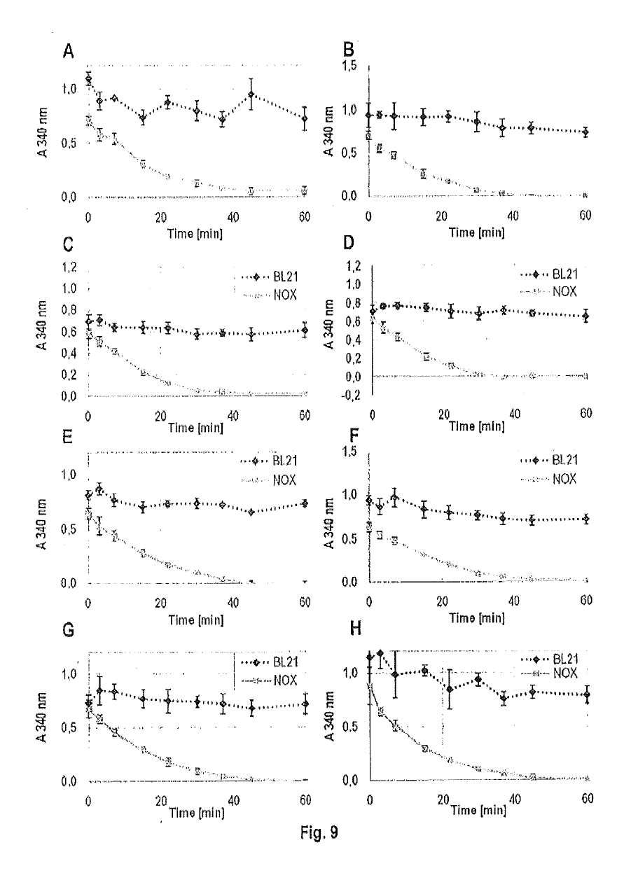

FIG. 9 shows the results for the NOX whole-cell biocatalyst, which had been stored for more than 7 weeks at −70° C. The activity assay was carried out with cells before storage [A] and cells that had been stored for one week [B], two weeks [C], three weeks [D], four weeks [E], five weeks [F], six weeks [G] and seven weeks [H].

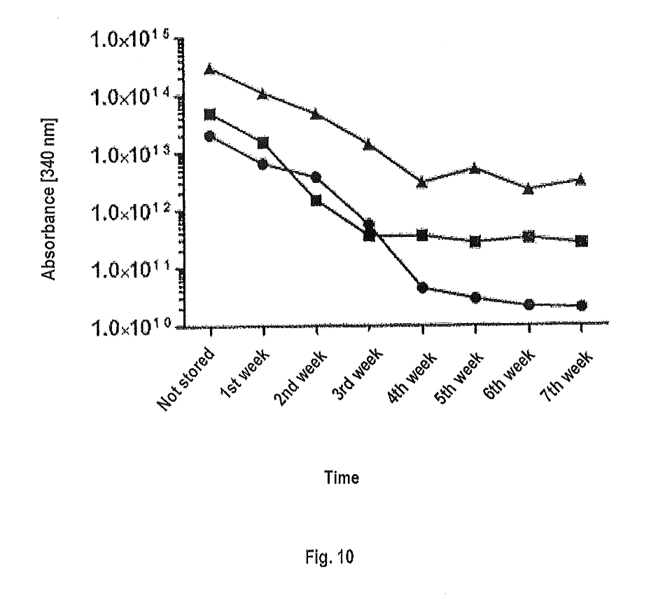

FIG. 10 shows the determination of the cell count in samples that had been stored for stability assays for more than seven weeks. This shows the decrease in cell count in samples that were stored at −18° C. (▪), as well as the decrease in cell count in samples that were stored at +8° C. (•), and samples that were stored at −70° C. (▴). For determining the cell count, 50 μl was taken from a stored partial sample and was used for preparing a series of dilutions. 50 ml of each of the 10⁻⁹ and 10⁻¹⁰ dilutions was plated out on LB-agar plates containing 30 mg/l kanamycin. The plates were incubated at 37° C. overnight, and the colonies were counted the next day. The cell count/ml was calculated on the basis of the number of colony-forming units.

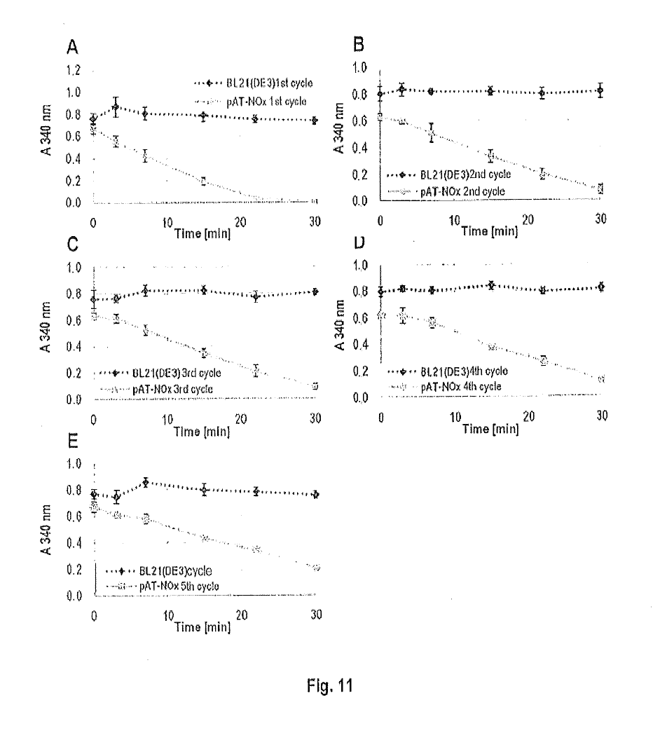

FIG. 11 shows the recyclability of the NOX whole-cell biocatalyst in an assay that was repeated cyclically five times. ♦=host strain E. coli BL21(DE3), ▪=E. coli BL21(DE3) pAT-NOx. In the narrower sense, the photometric determination of the decrease in NADH concentration in the first [A], second [B], third [C], fourth [D] and fifth [E] cycle is shown.

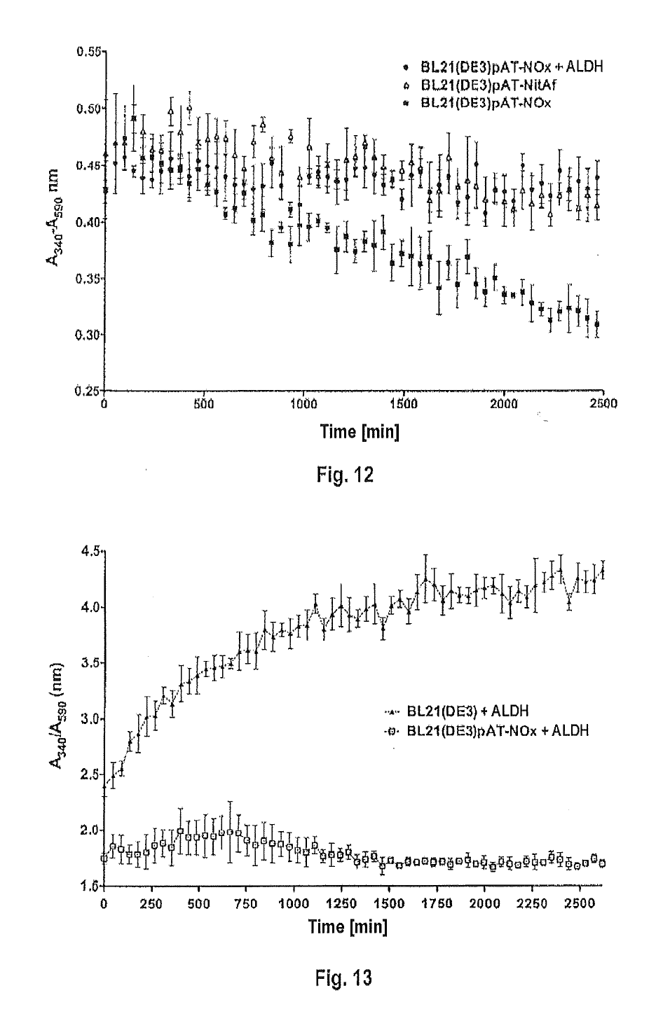

FIG. 12 shows the regeneration of NAD⁺ using the biocatalyst E. coli BL21(DE3) pAT-NOx, in the narrower sense the NADH conversion by the whole-cell biocatalyst E. coli BL21(DE3) pAT-NOx (▪), and in the regeneration system (•). E. coli BL21(DE3) pAT-NifAf (Δ) was used as control. The extinction of NADH was monitored at 340 nm. The extinction of the cells was monitored at 590 nm. Evaluation was carried out by calculating the difference E₃₄₀-E₅₉₀. ALDH 60 mU, cells OD 1, reaction started with 200 μM NADH.

FIG. 13 shows the regeneration of NAD⁺, in the narrower sense the concentration of NADH as a function of the extinction at 340 nm over time, in particular the NADH concentration during the ALDH reaction or during the ALDH reactions using E. coli BL21(DE3) pAT-NOx. The NADH concentration was normalized by calculating the quotient E₃₄₀/E₅₉₀ relative to the cell count. ALDH 30 mU, E. coli BL21(DE3) pAT-NOx OD 5, E. coli BL21(DE3) OD 1, reaction started with 400 μM NAD⁺.

### EXAMPLES

It is possible to detect the surface localization of a passenger, in this case of NADH oxidase, by incubating the bacteria in the presence of serine protease, trypsin or protein kinase K. As trypsin and proteinase K are unable, owing to their size, to pass through the outer membrane of E. coli, the incubation of whole cells in the presence of these proteases has the effect that only the proteins that are located on the surface of the outer membrane are digested. Therefore a passenger located on the surface is digested, whereas protein constituents integrated into the membrane are protected against enzyme attack and therefore remain intact. This can be confirmed by SDS-PAGE. The orientation of the enzyme was evaluated on the basis of whole-cell digestion. FIG. 2 shows the isolated outer membranes of the E. coli strain UT5600(DE3) pAT-NOx after induction of expression with IPTG as well as isolated outer membranes after whole-cell digestion. After the induction with IPTG, the expected fusion protein of about 100 kDa could be detected in the outer membrane (track 3). The digestion of whole cells with trypsin led to a decrease in intensity of the corresponding bands compared to the bands representing OmpF/C and OmpA of initially roughly equal intensity and thus confirmed that the protein is oriented toward the outside of the membrane (tracks 4 and 5). It could be demonstrated that NADH oxidase is displayed on the surface of E. coli using the autodisplay system.

### Example 2

### Establishing the Growth and Test Conditions for Monitoring NADH Oxidase Activity

The activity of NADH oxidase was determined with a photometric assay. NADH shows two extinction maxima, namely at the wavelengths of 260 nm and 340 nm, whereas NAD⁺ only shows one maximum, at 260 nm. This difference can be exploited for monitoring the oxidation of NADH to NAD⁺ photometrically. FAD was added to the nutrient media or buffers used, to allow proper folding of the enzyme as well as the uptake of FAD.

Culture of the NOX Biocatalyst

20 ml of LB medium (15 μg/ml kanamycin) was inoculated with 10 μl of glycerin culture of the corresponding strain and was incubated overnight at 37° C. and 200 revolutions per minute. For setting up the main culture, 100 ml of LB medium (15 μg/ml kanamycin, 10 μM EDTA, 10 mM mercaptoethanol) was inoculated with 2.5 ml of the overnight culture. At OD₅₇₈=0.6, 50 ml of the culture was induced with 1 mM IPTG. In the case of NADH oxidase, in addition 10 μM FAD was added. The cultures were induced for 1 h, 4 h or 20 h at 30° C. and 200 revolutions per minute. The other half of the culture was not induced, but otherwise was treated in exactly the same way as the induced culture.

Preparing the Cells for the NADH Oxidase Assay

Following the induction, the cells were first stored on ice for 15 minutes and were then separated as sediment by centrifugation for 10 min at 5000 revolutions per minute. The sediment was washed twice in 20 ml of 0.1 M potassium phosphate buffer, pH 7.5, 10 μM FAD. The OD of the cells was then adjusted to 50 with the same buffer.

NADH Oxidase Assays

The assay for detecting NADH oxidase activity was carried out with 1 ml samples, which contained 0.1 M potassium sulfate buffer, pH 7.5, 1 mM DTT and 100 μM NADH as substrate. The cells were used in the assay at a final OD₅₇₈ of 1. After incubation for one hour at room temperature, the cells were removed from the samples by centrifugation. The spectra of the supernatants were recorded at wavelengths between 200 and 400 nm.

First, for both strains, the expression of the NADH oxidase gene was induced for 1 h. The UV spectra in FIG. 3 show that it was impossible to detect enzyme activity under these conditions, since the NADH had already been oxidized by host cells that lacked the plasmid, which led to disappearance of the extinction maximum at 340 nm. The enzyme added is either taken up by the cells or transformed by enzymes secreted by the cells. Both effects can be explained by the high metabolic rate that the cells display in the exponential growth phase, as was found for the cells here.

In order to move the cells into the stationary growth phase and thus minimize their primary metabolism, the induction of expression was first prolonged to 4 h. It was found that the added NADH had once again been oxidized by the host cells (FIG. 4). However, the NADH-oxidizing activity of cells that contained pAT-NOx exceeded the activity of host cells that lacked the plasmid. Moreover, a quantitative difference could be detected between uninduced and induced cells.

After extending the induction time to 20 h, NADH oxidation by host cells that lacked the plasmid could no longer be detected, which can be seen from the fact that in the case of the negative control without cells, the extinction maximum of the NADH is present as before, as is shown in FIG. 5. It was now possible, for these so-called “quiescent cells” (cells in the stationary phase), to detect the oxidation of the added NADH by the NADH oxidase located on the surface. Many differences between E. coli BL21 (DE3) and E. coli UT5600 (DE3) are apparent. In the case of E. coli BL21(DE) pAT-NOx, uninduced cells convert about half of the supplied NADH to NAD⁺, whereas induced cells convert all available NADH. The activity of the uninduced cells can be explained on the basis that the T7 system used for the expression of NADH oxidase is not “dense”, i.e. the repression of the T7-promoter is incomplete, which even in the uninduced state leads to low expression of the gene by T7-polymerase. In contrast, E. coli UT5600 (DE3) shows no activity in the uninduced state, but induced cells only convert about half of the NADH.

These results prove that the flavoprotein-NADH oxidase of Lactobacillus brevis can be expressed on the surface of E. coli in its active form, using the autotransporter system. The subsequent incorporation of FAD in the apoenzyme seems to proceed properly and only requires the addition of FAD. All measurements so far were of a qualitative nature, for setting up the assay and for finding conditions that allow the activity of the enzyme to be monitored.

For quantitative monitoring of the enzyme activity by measuring NADH oxidation over time, the optical assay was transferred to microtiter plates, so as to permit parallel observation of as many samples as possible.

Culture of the Cells in Minimal Medium (M9)

10 ml of M9 medium (15 μg/ml kanamycin) was inoculated with 20 μl of glycerin culture of the corresponding strain and was incubated overnight at 37° C. and 200 revolutions per minute. The whole overnight culture was used for inoculating the main culture, 160 ml of M9 (15 μg/ml kanamycin). After initial growth of the cultures for 7 h, 1 mM IPTG and 0.2% lactose, as carbon source, were added to half of the culture. In addition, 10 μM FAD was added for the NADH oxidase. Induction was carried out for 16 h at 30° C. and 200 revolutions per minute. IPTG was not added to the other half of the cultures, but otherwise these cultures were treated in exactly the same way.

Preparing the Cells for Activity Assays

After the induction, the cells were first stored on ice for 15 minutes and then separated as sediment by centrifugation for 10 min at 5000 revolutions per minute. The sediments were washed twice in 20 ml of 0.1 M potassium phosphate buffer, pH 7.5, 10 μM FAD. Then the cells were adjusted with the same buffer to OD₅₇₈=10.

NADH Oxidase Assay on Microtiter Plates

The NADH oxidase assay for 96-well microtiter plates was conducted in samples of 240 μl per well in 0.1 M potassium phosphate buffer. The cells were used in amounts such that an OD₅₇₈ of 1 was achieved in the assay. For starting the reactions, 200 μM NADH was added. After incubation for 0, 3, 7, 15, 30, 45 or 60 minutes at room temperature, the assay was observed in a microtiter plate at 340 nm (Mithras, Berthold Technologies). To avoid sedimentation of the cells, the plate was shaken before measuring the extinction. A cell suspension with the corresponding OD in buffer was used as reference.

Based on a series of tests, production of the NADH oxidase whole-cell catalyst could be optimized so that i) activities were higher, ii) the difference between induced and uninduced cells was increased and iii) for the nonsense controls there was only low activity. In particular, it was found that the culture that is used for measuring the NADH oxidase must be in an optimal state. FIG. 6 shows the results of an NADH oxidase assay that was performed under standard conditions, as described in the “Methods” section.

Various factors connected with the growth and test conditions are responsible for this improved enzyme activity. Already with a view to use on a larger scale, the growth of the cells was modified so that instead of the complex LB medium, defined M9 medium was used. During the induction, lactose was added as carbon source, and the cells were induced earlier, at an OD₅₇₈ of about 0.35. Later induction, usually at 0.6, has the result that excessively long growth in the M9 medium is necessary, which leads to spontaneous cell lysis, with the effect that both the nonsense controls and the uninduced controls oxidize the NADH supplied as substrate. This is very probably due to enzymes that are released as a result of the cell lysis.

The NADH oxidase assay was now performed and observed in the microtiter plate directly in the cell suspension, as described above. This makes handling much easier and the data are more reproducible.

However, determination of absolute values of enzyme activity is not possible. The activity of enzymes determined with a photometric method is calculated from the Beer-Lambert law. To be able to apply this equation, the layer thickness of the cuvette or of the 96-well plate must be known. This parameter cannot be determined in the present test setup. Therefore the activity of the NADH oxidase was determined with a photometer, using cuvettes with a defined layer thickness of 1 cm. Consequently, the Beer-Lambert law can be applied. At a temperature of 30° C., 12.23 mU/ml was found for the activity in the LB medium. The activity at 30° C. in the M9 medium was somewhat higher, namely 16.38 mU/ml. In the work of Hummel et al. (2003) the activity of an enzyme purified in a simple manner was given as 10-15 U/mg.

### Example 3

### Testing the Stability and Storage Capability of the NADH Oxidase Whole-Cell Catalyst

For investigating the storage capability and stability of the NADH oxidase whole-cell catalyst, induced cells were stored at 8° C. in the refrigerator or at ˜18° C. or ˜70° C. in the freezer. These cells were checked weekly by the NADH oxidase assay with samples in triplicate.

The cells intended for storage were, after the induction of the gene expression, washed twice with potassium phosphate buffer (0.1 M, pH 7.5), adjusted in this buffer to OD₅₇₈=10, divided up into partial samples and put in storage for eight weeks at +8° C. If cells were to be stored at −18° C. or −70° C., 20% glycerin was added to the freezing buffer. For activity assay, the cells, in 200-μl samples, were diluted with the aforementioned buffer to OD₅₇₈=1, and then 0.2 mM NADH was added to start the reaction. The oxidation of NADH to NAD was monitored in samples in triplicate by measuring the decrease in extinction at λₘₐₓ=340 nm. To take account of the effect of the cell lysis occurring during storage, the host strain E. coli BL21 (DE3) was stored in the same way and underwent the same type of activity assay as the whole-cell biocatalyst BL21 (DE3) pAT-NOX.

The graphs in FIG. 7 show the decrease in NADH concentration in cell suspensions of varying age, stored at 8° C. As expected, the host strain E. coli BL21(DE3), which was used as control, does not show any NADH activity before storage. However, for these host cells, after just one week of storage, a clear decrease in NADH concentration is found in the assay, and this decrease becomes more pronounced the longer the storage time. This presumed NADH oxidase activity can be explained by the lysis of bacterial cells, which based on experience begins relatively quickly when cells are stored at 8° C. Cells stored at this temperature in the refrigerator already show increased viscosity in solution after a few days and cannot be resuspended completely. This increased viscosity can be attributed to release of chromosomal DNA during the cell lysis. In addition, as a result of the lysis of the cells, enzymes are released which evidently oxidize the NADH used as substrate in the assay. This effect can also be observed in the case of storage of the NADH oxidase whole-cell catalyst E. coli BL21(DE3) pAT-NOX and a subsequent activity assay. When the storage time increases, the NADH oxidation that is brought about by the lysis of the cells in the refrigerator will exceed the activity of the catalyst measured before storage. Therefore it cannot be recommended to keep the NADH oxidase whole-cell catalyst in the refrigerator.

When cells are stored at −18° C., the situation is different (FIG. 8). For the host cells used as negative control, after storage in a chest freezer at −18° C. there is no consequent oxidation of NADH, whereas the NADH oxidase whole-cell catalyst, even after 8 weeks, still displays NADH oxidase activity, which—in the context of a normal distribution—is comparable to the initial activity.

With storage of the whole-cell biocatalyst at −70° C. for weeks, no significant decrease in activity is observed (FIG. 9). Lysis of host cells can in this case also be ruled out. Therefore it is recommended to store the whole-cell biocatalyst at −70° C.

Meanwhile, in parallel with the activity of the biocatalyst, the live cell count was determined. The results are shown in FIG. 10. With storage at +8° C., the live cell count decreases within the first four weeks by three orders of magnitude. With storage lasting a further three weeks, the live cell count then remains relatively constant. This shape of the curve is also observed with storage of the biocatalyst at −18° C. or −70° C. However, in this case the decrease in live cell count during the first four weeks is only two orders of magnitude. The decrease in live cell count is not correlated with the activity of the biocatalyst over a period of seven weeks. The live cell count decreases with storage at −18° C. or −70° C., but the activity remains roughly constant. However, this test shows that storage in the chest freezer (−18° C. or −70° C.) is preferable to the storage at +8° C. in the refrigerator.

### Example 4

### Investigation of the Recyclability of the Whole-Cell Biocatalyst

The recyclability of the whole-cell catalyst E. coli BL21(DE3) pAT-NOx was determined with respect to conversion of NADH over a period of 30 minutes in 5 cycles. For this assay, following the induction of the gene expression, the cells were washed twice with potassium phosphate buffer (0.1 M, pH 7.5) and were resuspended in this buffer to a final OD₅₇₈ of 10. For determining the activity, cells in 200-μl samples were diluted with the aforementioned buffer in the microtiter plate to an OD₅₇₈ of 1, and then 0.2 mM NADH was added to start the reaction. The oxidation of NADH to NAD was monitored in samples in triplicate by measuring the decrease in extinction at λₘₐₓ=340 nm. For recycling, the cells were centrifuged down after completion of the reaction and were resuspended in 160 μl potassium phosphate buffer. Once again, 0.2 mM NADH was added to start the reaction, and the oxidation was monitored photometrically. This procedure was repeated for a total of 5 cycles. To take account of the influence of the cell lysis possibly occurring during the treatment, the host strain E. coli BL21 (DE3) as negative control was submitted to the same assay as the whole-cell biocatalyst BL21 (DE3).

Host cells that lacked the biocatalyst were used as control. As can be seen from FIG. 11, the host cells do not convert any NADH. In the first cycle (A), the whole-cell biocatalyst converts almost 100% of the NADH present within 30 minutes. In the further cycles, the conversion and the conversion rate decrease. In the last cycle (5th cycle, D) 30% less NADH is converted within the incubation time. The centrifugation of the bacterial suspension might be responsible for the decrease in activity in subsequent cycles. The centrifugal force represents a mechanical stress for the cell and especially for the molecules expressed on its surface.

### Example 5

### Regeneration of NADH to NAD⁺ Using the Whole-Cell Biocatalyst E. coli BL21 (DE3) pAT-NOx

This test was carried out in order to investigate whether the whole-cell biocatalyst E. coli BL21 (DE3) pAT-NOx can be used as regeneration system for NAD⁺-reducing enzymes. This was tested using a system that comprises the whole-cell biocatalyst and an NAD⁺-reducing enzyme, aldehyde dehydrogenase (ALDH). ALDH belongs to a group of enzymes that oxidize aldehydes to the corresponding carboxylic acids and therefore reduce NAD⁺ to NADH. The NADH concentration was monitored photometrically.

As can be seen from FIG. 12, as expected, the NADH concentration in the sample containing the whole-cell biocatalyst E. coli BL21(DE3) pAT-NOx decreases continuously. In contrast, an equilibrium is established between NAD⁺ and NADH if the recombinant enzyme ALDH is added to the biocatalyst, as the NAD⁺ that is reduced continuously to NADH by the ALDH is regenerated by the biocatalyst E. coli BL22(DE3) pAT-NOx. Therefore this system is in equilibrium. E. coli BL21 (DE3)-NitAf served as so-called nonsense-control. These cells display on their surface the nitrilase from A. faecalis, which is unable to oxidize NADH, as can also be seen from FIG. 12.

FIG. 13 shows, in the upper half of the graph, the NADH increase mediated by ALDH. Regarding addition of the biocatalyst E. coli BL21(DE3) pAT-NOx for regenerating NAD⁺, the host strain E. coli BL21(DE3) had already been added to this reaction, so as to be able to standardize the NADH concentration (determined as extinction at 340 nm) with respect to the cell count. Following addition of the biocatalyst E. coli BL21(DE3) pAT-NOx to the ALDH reaction, no further increase in NADH could be found over time, as the NADH formed by ALDH was continually reoxidized to NAD⁺. Therefore this system is in equilibrium.

### DETAILS OF SOURCES

Insofar as reference is made here to various documents of the prior art, these documents in their entirety form part of the present description by reference. The complete bibliographic details for the cited documents of the prior art are given below:

Sanjust et al. (1997), Biochem Mol Biol Int (3), 555-62.

Hummel and Riebel (2003), Biotechnol Lett. (1), 51-4.

Ahmed and Claiborne (1992), J. Biol. Chem. 267(36): 25822-9.

El-Zahab et al. (2004), Appl Biochem Biotechnol., 117(3), 165-74.

Lee and Ping (2007), Biotechnology Adv 25, 369-384.

Jose and Meyer (2007), Microbiol Mol Biol Rev 71 (4), 600-19.

Jose (2006), Appl. Microbiol. Biotechnol. 2006, 69, 607-614.

Jose et al. (2002), J. Biotechnol. 2002, 95, 257-268.

Jose et al. (1995), Mol. Microbiol., (2), 378-80.

Ausubel et al. (1995), Current Protocols in Molecular Biology, Wiley Interscience Publishers.

Henderson et al. (2004), Microbiol. and Molecular Biology Reviews, 68(4), 692-744.

Jose and von Schwichow (2004), ChemBioChem, 5, 491-499. Banerjee et al. (2002), Appl. Microbiol. Biotechnol. 2002, 60, 33-44.

Sarborsky and Ogletree (1974), Biochem Biophys Res Commun., 61(1): 210-6.

“Export Systems for recombinant proteins”, International Patent Application WO 97/35 022.

Niewert et al. (2001) Diarrhea. Clin. Diagn. Lab. Immunol. (1): 143-149; 9.

The features of the present invention, which are disclosed in the description, in the sequence listing, in the claims and/or in the drawings, can be legally relevant both alone and in any combination for implementing the invention in its various forms.

1-14. (canceled)

15. A nucleic acid molecule, comprising the following constituents:
(1) a segment coding fora signal peptide,
(2) a segment comprising a heterologous redox factor-regenerating polypeptide,
(3) optionally a segment coding for a protease recognition site,
(4) a segment coding for a transmembrane linker, and
(5) a segment coding for a transporter domain of an autotransporter or of a variant thereof,
wherein the redox factor-regenerating polypeptide is a polypeptide suitable for regenerating redox factors selected from the group comprising NADH, NAD⁺, NADPH, NADP⁺, FADH₂ and FAD.

16. The nucleic acid molecule as claimed in claim 15, wherein the redox factor-regenerating polypeptide comprises one or more flavin cofactors.

17. The nucleic acid molecule as claimed in claim 15, wherein the redox factor-regenerating polypeptide is selected from the group comprising NADH oxidase, formate dehydrogenase, glucose dehydrogenase.

18. The nucleic acid molecule as claimed in claim 16, wherein the redox factor-regenerating polypeptide is selected from the group comprising NADH oxidase, formate dehydrogenase, glucose dehydrogenase.

19. The nucleic acid molecule as claimed in claim 17, wherein the redox factor-regenerating polypeptide is selected from the group comprising the NADH oxidases of Lactobacillus brevis, Thermus thermophilus, Thermus aquaticus, Brevibacterium sp. KU139 and Streptococcus faecalis, preferably the NADH oxidase of Lactobacillus brevis, and variants thereof.

20. The nucleic acid molecule as claimed in claim 15, wherein the transporter domain of an autotransporter is selected from the group comprising Ssp, Ssp-h1, Ssp-h2, PspA, PspB, Ssa1, SphB1, AspA/NalP, VacA, AIDA-I, IcsA, MisL, TibA, Ag43, ShdA, AutA, Tsh, SepA, EspC, EspP, Pet, Pic, SigA, Sat, Vat, EpeA, EatA, EspI, EaaA, EaaC, pertactin, BrkA, Tef, Vag8, PmpD, Pmp20, Pmp21, AgA1 protease, App, Hap, rOmpA, rOmpB, ApeE, EstA, Lip-1, McaP, BabA, SabA, AlpA, Aae, NanB and variants thereof.

21. The nucleic acid molecule as claimed in claim 15, wherein the redox factor-regenerating polypeptide is oxygen-sensitive.

22. A polypeptide encoded by a nucleic acid molecule as claimed in claim 15.

23. A cell that expresses a polypeptide as claimed in claim 22 on its surface.

24. A cell that has been transformed using a nucleic acid molecule as claimed in claim 15.

25. A membrane fraction obtainable from the cell as claimed in claim 23.

26. A membrane fraction obtainable from the cell as claimed in claim 24.

27. A method of regenerating a redox factor, comprising
contacting the polypeptide as claimed in claim 22 with one or more substrates of the redox factor-regenerating polypeptide,
wherein the one substrate or the several substrates comprises or comprise the redox factor.

28. A method of regenerating a redox factor, comprising
contacting the membrane preparation as claimed in claim 25 with one or more substrates of the redox factor-regenerating polypeptide,
wherein the one substrate or the several substrates comprises or comprise the redox factor.

29. A method of regenerating a redox factor, comprising
contacting the cell as claimed in claim 23 with one or more substrates of the redox factor-regenerating polypeptide,
wherein the one substrate or the several substrates comprises or comprise the redox factor.

30. A method of producing a cell that displays a redox factor-regenerating polypeptide on its surface, comprising the following steps:
(a) introducing the nucleic acid as claimed in claim 15 into a cell,
(b) optionally contacting the cell with one or more redox-reactive prosthetic groups.

31. A method of producing a product of a redox factor-dependent enzyme, comprising
contacting the redox factor-dependent enzyme with one or more of its substrates in the presence of the cell as claimed in claim 23,
wherein the cell as claimed in claim 23 comprises a polypeptide that regenerates the redox factor on which the redox factor-dependent polypeptide depends.

32. A method of producing a product of a redox factor-dependent enzyme, comprising:
contacting the redox factor-dependent enzyme with one or more of its substrates in the presence of the membrane preparation as claimed in claim 25,
wherein the membrane preparation as claimed in claim 25 comprises a polypeptide that regenerates the redox factor on which the redox factor-dependent polypeptide depends.

33. A method of producing a product of a redox factor-dependent enzyme, comprising:
contacting the redox factor-dependent enzyme with one or more of its substrates in the presence of the polypeptide as claimed in claim 22,
wherein the polypeptide as claimed in claim 22 comprises a polypeptide that regenerates the redox factor on which the redox factor-dependent polypeptide depends.
